(19) United States
(12) Patent Application Publication
(10) Pub. No.: US 2019/0231569 A1
(43) Pub. Date: Aug. 1, 2019

(54) DRIVERS AND SET FOR THE INSERTION OF CONNECTABLE IMPLANTS

(71) Applicants: ID Nest Medical, Strasbourg (FR); BS MEDICAL TECH INDUSTRY, Niederroedern (FR)

(72) Inventors: Bertrand Basch, Soufflenheim (FR); David Contassot, Strasbourg (FR)

(21) Appl. No.: 16/335,104

(22) PCT Filed: Oct. 27, 2017
(86) PCT No.: PCT/FR2017/052969

§ 371 Date: Mar. 20, 2019

(30) Foreign Application Priority Data
Oct. 27, 2016 (FR) 1660463

(51) Int. Cl. A61F 2/954; A61F 2/97; A61F 2/07

(52) U.S. Cl. CPC A61F 2/954; A61F 2002/061; A61F 2/07; A61F 2/97

(57) ABSTRACT

The invention relates to a set of drivers for the insertion and connection of implants in conduits exhibiting a bifurcation, a main driver having an internal sheath provided with an atraumatic tip and an axial hole, an external sheath, a main implant disposed between the sheaths, the external sheath being provided with a groove along a part of its length, the internal sheath having a lateral hole facing said groove, said main implant having a window aligned with the lateral hole, the main driver having an extractable complementary sheath, a first end of which passes through the lateral hole, the window and the groove, a secondary driver having an internal secondary sheath provided with a secondary atraumatic tip which has a secondary axial hole, an external secondary sheath, a secondary implant disposed between the secondary internal and external sheaths, a main guide intended to be fitted in the axial hole in order to guide the main driver, a secondary guide passing through the bifurcation in order to be fitted in the complementary sheath in order to position the main driver and/or in order to guide the secondary driver.

### TECHNICAL FIELD

The present description relates to the general technical field of the insertion of connectable implants in all circulation conduits of a bodily fluid exhibiting one or more bifurcations. Connectable should be understood as implants able to be mechanically linked, once inserted into the conduits of the bifurcation.

The description more particularly relates to means intended for example for the insertion of implants, such as connectable stents, at the confluence of the iliac veins, or connected endoprostheses at the level of aortic arch. These means are called “drivers”, or “insertion devices or systems”. The implants may for example be self-expandable or balloon-expandable stents or in general any existing type of stents.

The described embodiments is also applicable in urology.

These means are for example intended to insert connected implants in a cavity of a living being, and particularly in the circulatory, and especially arterial and venous, system, in a region where the corresponding vessel has collateral branches requiring the maintenance of their perfusion.

In this case, a first device allows the insertion of the parent tubular implant into a blood circulation conduit such as the aorta.

A second device allows the deployment of branches with any shape and characteristic, for example to allow the vascularization of the internal iliac artery at the thoracoabdominal aorta and of the left common carotid artery, the left subclavian artery and the brachiocephalic trunk at the aortic arch and lastly of coronary arteries at the aortic root.

Likewise, such a device applies to all of the venous-type bifurcations such as the inferior vena cava and its iliac and renal branches, and the superior vena cava and its branches.

Other applications are also possible in any other circulation conduit for a bodily fluid exhibiting one or more bifurcations.

Such means are generally used for the insertion of implants in the case of vascular disease such as aneurysms, dissections, occlusive diseases and compressive syndromes.

The implantations are generally done using endoluminal techniques. These minimally invasive techniques lead to lower patient mortality and morbidity rates, as well as decreased operating times.

### BACKGROUND

Known for example from document EP 1 648 343 is a system for treating a vascular bifurcation. The described system includes a catheter with two branches making it possible to insert a stent into each of the conduits of the bifurcation. Such a system has the disadvantage of being provided with a large number of component elements that must be inserted into one of the conduits. The assembly of such a system is therefore complex in terms of manufacturing. Furthermore, this has a non-negligible impact on its bulk, which is not desirable. The use of such a complex system makes the surgical procedure more complex, which may result in incorrect manipulations or less reliable positioning of the implants. The placement of guides is also not done as a first-line treatment, and remains complex and random due to the obstruction of the conduit by the main stent that has already been implanted.

Also known from document US 2012/0271410 A1 is a system for inserting two stents at a bifurcation. The system includes a cover element realizing the mechanical link and preassembly of the stents before insertion. This system is therefore not suitable for connecting implants that are not provided with cover elements. Furthermore, this system comprises only one driver delivering a main stent and a branch stent and the cover elements. The dimensional bulk necessary for such a driver is therefore unfavorably increased. The use of a single driver, which also makes it possible to position guides in the conduits, alters the maneuverability of the various elements, in particular the stents and the sheaths, during the insertion operation. Indeed, any movement of one stent necessarily causes a movement on the other stent. The stent insertion operation is thus made more complicated, less precise and therefore less reliable.

### SUMMARY OF THE DESCRIPTION

The aim of the presently described embodiments is in particular to overcome the disadvantages of the state of the art and to propose a new insertion assembly making it possible to produce, in situ, the connection between a primary tubular implant and at least one secondary tubular implant simply, precisely and reliably.

Another aim of the described embodiments is to propose a new method for positioning and connecting implants, the implementation of which is simple and reliable.

The purposes assigned to the described embodiments are achieved using a main driver having at least one main implant for the insertion of said at least one implant connectable to another implant, at a bifurcation between a main conduit and a secondary main conduit of circulation for a bodily fluid, said main driver having a distal end D and a proximal handling end P having a fixed point,

characterized in that:
said main driver has an internal sheath provided at its distal end D with an atraumatic tip with an axial hole communicating with a longitudinal tube formed by said internal sheath,
said main driver having an external sheath,
said at least one main implant being disposed between the external and internal sheaths,
the main external sheath being provided with a longitudinal groove or slit extending along a part of the length,
the main internal sheath having a lateral hole emerging facing said longitudinal groove or slit,
said main implant having at least one window aligned with the lateral hole,
and said main driver having at least one complementary sheath, a first end of which passes through the lateral hole, the window of the main implant and the longitudinal groove or slit, and a second end of which comes out near the proximal end P, said complementary sheath being intended to be passed through by a guide.

According to one example embodiment of the driver, the main external sheath is mounted sliding on the main internal sheath and blocked in sliding by the atraumatic tip on the one hand and by at least one removable stop on the other hand, said longitudinal groove or slit being open at its distal end near the atraumatic tip.

According to one example embodiment of the driver, the main external sheath is made of a peelable material.

According to one example embodiment of the driver, the complementary sheath is extractable from the main driver and allows the insertion of a secondary guide into said main driver and makes it possible to provide the passage in the window of the main implant and the passage of the lateral hole of the internal sheath.

The purposes assigned to the described embodiments are also achieved owing to an assembly for the insertion and connection of at least two implants in main and secondary circulation conduits for a bodily fluid, the main conduit having, with the secondary conduit, a bifurcation, characterized in that it comprises:
a main driver as described above,
a secondary driver, independent of the main driver, having a distal end D and a proximal handling end P having a fixed point,
said secondary driver having an internal secondary sheath provided at its distal end D with a secondary atraumatic tip with a secondary axial hole communicating with a longitudinal tube formed by said internal secondary sheath, an external secondary sheath, at least one secondary implant disposed between the external and internal secondary sheaths.

According to one example embodiment, the assembly comprises a main guide pre-implantable in the main conduit and intended to engage in the axial hole for guiding purposes of the main driver.

According to one example embodiment, the assembly comprises a secondary guide pre-implantable in the main conduit and in the secondary conduit while passing through the bifurcation, to engage in the complementary sheath for positioning purposes of the main driver and/or for guiding purposes of the secondary driver.

According to one example embodiment of the assembly, the secondary atraumatic tip of the secondary driver is a double-cone tip.

According to one example embodiment of the assembly, the secondary external sheath is mounted sliding on the secondary internal sheath and blocked in sliding by the secondary atraumatic tip on the one hand and by at least two successive and removable stops A, B on the other hand, said stops A, B ensuring to the gradual release of the secondary implant as well as its connection on the main implant.

According to another example embodiment of the assembly, the secondary external sheath is mounted sliding on the secondary internal sheath and blocked in sliding by the secondary atraumatic tip on the one hand and by three successive and removable stops A, B, C on the other hand.

According to one example embodiment of the assembly, the secondary implant is configured to have connection members to the main implant, which automatically form the connection with the main implant during the release and deployment of said secondary implant.

According to one example embodiment of the assembly, the secondary atraumatic tip has a curved distal end D.

According to one example embodiment of the assembly, the secondary atraumatic tip is made from a flexible material.

According to one example embodiment of the assembly, the implants are connected to the corresponding internal sheath by means of a retaining member making it possible to remove and/or reposition said implants in the driver if needed during the insertion operation.

The purposes assigned to the described embodiments are also achieved using a method for the insertion and in situ connection of two implants in main and secondary circulation conduits for a bodily fluid, the main circulation conduit having, with the secondary circulation conduit, a bifurcation, said method being carried out with an assembly according to the above description, characterized in that it comprises the following steps:
engaging a secondary guide in the secondary conduit,
introducing a gripping member into the main conduit, and attaching the secondary guide at the level of the bifurcation,
passing the end of the secondary guide in the main conduit and extracting one end thereof from the main conduit,
engaging a main guide in the main conduit,
engaging the end of the secondary guide, exiting from the main conduit, into the first end of the complementary sheath of the main driver,
engaging the end of the main guide, exiting from the main conduit, into the axial hole of the main driver,
removing the complementary sheath from the main driver,
introducing the main driver and positioning it at the bifurcation using the main guide,
sliding the external sheath over the internal sheath to release the main implant and deploy the main implant,
introducing the secondary driver into the secondary conduit up to the level of the main implant using the secondary guide,
sliding the external secondary sheath over the internal secondary sheath to gradually release the secondary implant, deploy it and connect it to the main implant,
removing the main and secondary drivers as well as the main and secondary guides from the conduits.

According to one example embodiment, the method consists of sliding the external secondary sheath and using a system of removable stops to block the sliding of the external secondary sheath in successive positions corresponding to the gradual release of the secondary implant as well as its connection to the main implant.

According to one example embodiment, the method consists of repositioning the secondary implant (13) in the secondary driver (2) for a release and placement in another more suitable location in the secondary conduit (C2).

The assembly has the advantage of being easy to use and manipulate while guaranteeing remarkable reliability in the positioning of the implants. Indeed, using two separate drivers, inserted into the respective conduits via distinct incisions, makes it possible to manipulate each implant individually. This increases the precision of the release of the implants and the reliability of the connection between the implants.

Another advantage of the assembly lies in the use of each of the conduits of the bifurcation for the insertion of a driver. Each of the drivers, having only a small number of elements, may therefore have a small bulk and may simplify the surgeon's actions.

Another advantage of the assembly, and more particularly of the insertion and connection method, lies in obtaining a remarkable precision and reliability of the operating actions.

Another advantage of the driver or the assembly lies in the absence of sharp edges able to catch on the implants after they are deployed.

The manufacturing costs of the assembly are also reduced relative to the costs of the known systems. Furthermore, with an assembly, the implantation site is perfectly controlled, the procedure time is reduced and the operating risks are reduced.

### BRIEF DESCRIPTION OF THE FIGURES

Other advantages and particularities will also be apparent upon reading the appended drawings, which are given by way of illustrative and non-limiting examples wherein:

FIG. 1 is an illustration of an example embodiment of a main driver,

FIG. 2 is a partial enlargement of the main driver from FIG. 1,

FIG. 3 is an illustration of an enlarged detail from FIG. 1 or 2,

FIG. 4 is an illustration of the main driver of FIG. 1, ready to be used for a surgical procedure,

FIGS. 5 and 6 illustrate two other embodiments of the main driver,

FIG. 7 is an illustration of an example embodiment of a secondary driver of an assembly,

FIG. 8 is a partial enlargement of the secondary driver from FIG. 7,

FIG. 9 is an illustration of the secondary driver of FIG. 7, ready to be used for a surgical procedure,

FIG. 10 is an enlarged schematic illustration of the distal end of the secondary driver from FIG. 7, wherein the secondary implant is disposed compactly before insertion,

FIG. 11 illustrates the detail of FIG. 10 with the secondary implant released and deployed,

FIG. 12 illustrates the main driver with the main implant in a deployed state,

FIG. 13 is a partial illustration of an enlargement of FIG. 12,

FIGS. 13 to 25 schematically illustrate the insertion and connection method of the implants at a bifurcation of conduits using the assembly,

FIGS. 26 to 31 illustrate, with an anatomic localization, some of the steps illustrated in FIGS. 13 to 25,

FIG. 32 illustrates an example embodiment of the configuration of the proximal handling end of a secondary driver of the assembly, before the insertion of a secondary implant,

FIG. 33 schematically shows, in section, the distal end of the secondary driver in the configuration of FIG. 32,

FIGS. 34 to 39 illustrate the release steps of the secondary implant through respective illustrations of the proximal (FIGS. 34, 36, 38) and distal (FIGS. 35, 37, 39) ends of the secondary driver of the assembly, and

FIG. 40 shows an example embodiment of a connection between a main implant and a secondary implant, obtained using an assembly.

### DETAILED DESCRIPTION

The structurally and functionally identical elements shown in different figures keep the same numerical or alphanumerical reference.

The described embodiments relate to an assembly for the insertion and connection of at least two implants in main and secondary circulation conduits for a bodily fluid, in fluid communication via a bifurcation. The secondary circulation conduit is also called branch conduit. The bifurcation may have main C1 and secondary C2 conduits extending substantially orthogonally or with a determined inclination.

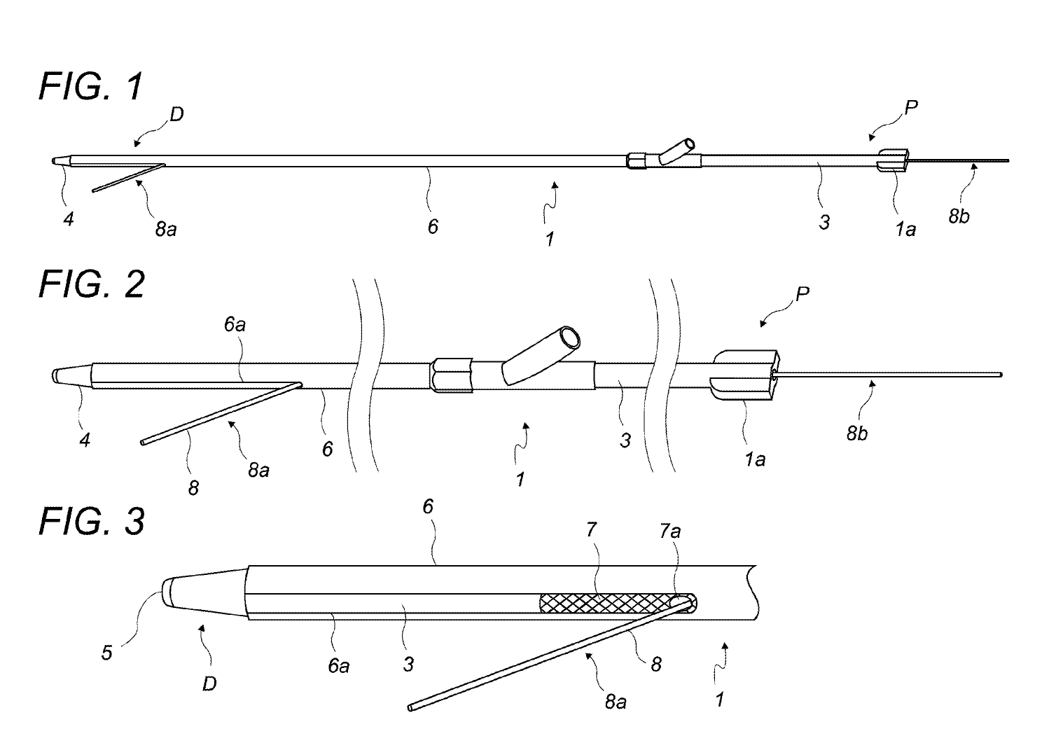

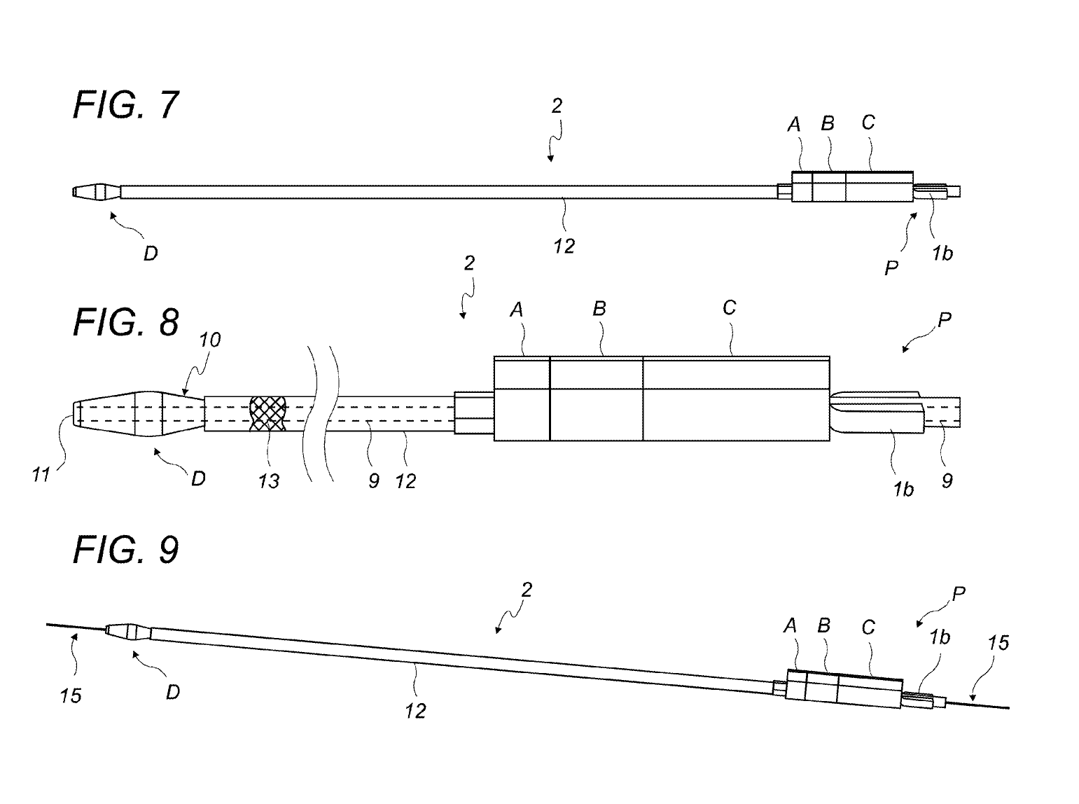

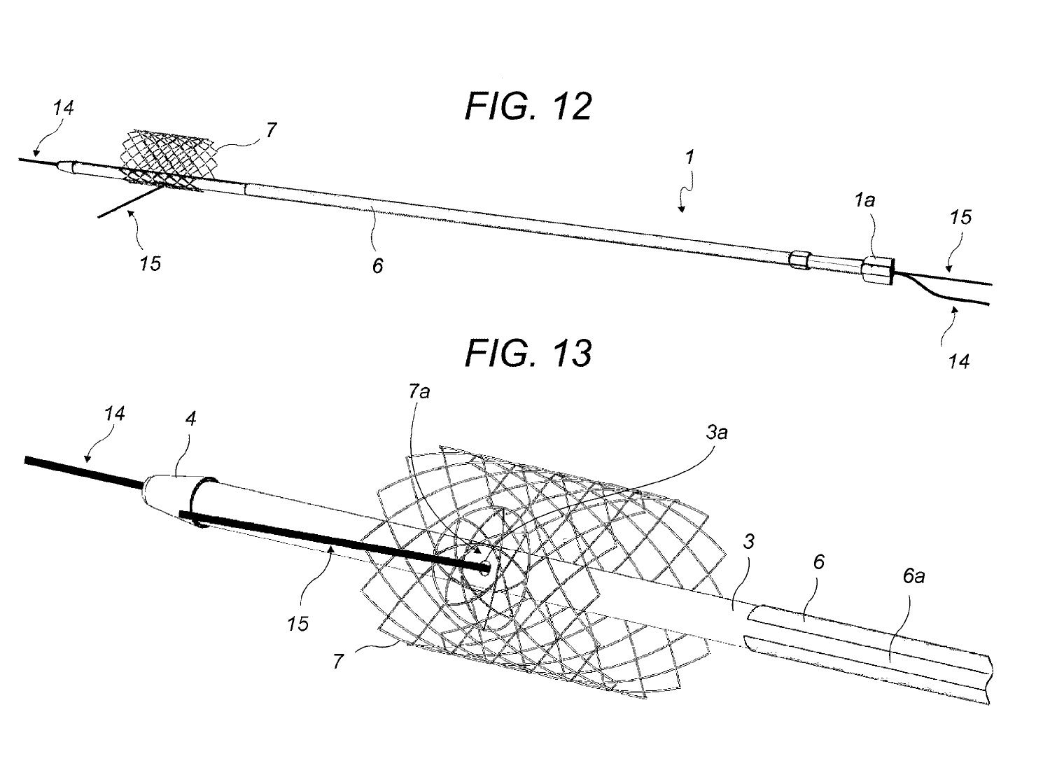

The assembly includes a main driver 1 and a secondary driver 2, the main conduit having a bifurcation with the secondary conduit. FIGS. 1 to 6, 12 and 13 are illustrations of example embodiments of the main driver 1, while FIGS. 7 to 11 are illustrations of an example embodiment of the secondary driver 2 of the assembly.

The main 1 and secondary 2 drivers each have a distal end D and a proximal handling end P having a fixed point 1a and 1b respectively.

The main driver 1 has an internal sheath 3 provided at its distal end with an atraumatic tip 4 with an axial hole 5 communicating with a tube formed by said internal sheath 3. The main driver 1 also has a main external sheath 6. At least one main implant 7 is disposed in a non-deployed manner between the external 6 and internal 3 sheaths.

The external sheath 6 is provided with a longitudinal groove or slit 6a in the form of an opening, on part of its length and open at its end near the atraumatic tip 4. Reference may also be made to FIGS. 3 and 13, showing the main implant 7, respectively not deployed and deployed after a removal by sliding of the main external sheath 6.

The internal sheath 3 advantageously has a lateral hole 3a, radial and opening facing said longitudinal groove or slit 6a. Reference may in particular be made to FIG. 13.

The main implant 7, mounted on the internal sheath 3, has at least one window 7a aligned with the aforementioned lateral hole 3a and with the longitudinal groove or slit 6a. Reference may for example be made to FIG. 3.

The main driver 1 also includes a complementary sheath 8, a first end 8a of which passes through the lateral hole 3a, the window 7a of the main implant 7 and the longitudinal groove 6a. A second end 8b of the complementary sheath 8 exits the main driver 1 near its proximal end P.

The secondary driver 2 has an internal secondary sheath 9, illustrated schematically in FIG. 8 and provided at its distal end D with a secondary atraumatic tip 10 with a secondary axial hole 11 communicating with an internal tube formed by the internal secondary sheath 9. The secondary driver 2 also has an external secondary sheath 12. At least one secondary implant 13 is disposed in a non-deployed manner between the external 12 and internal 9 secondary sheaths.

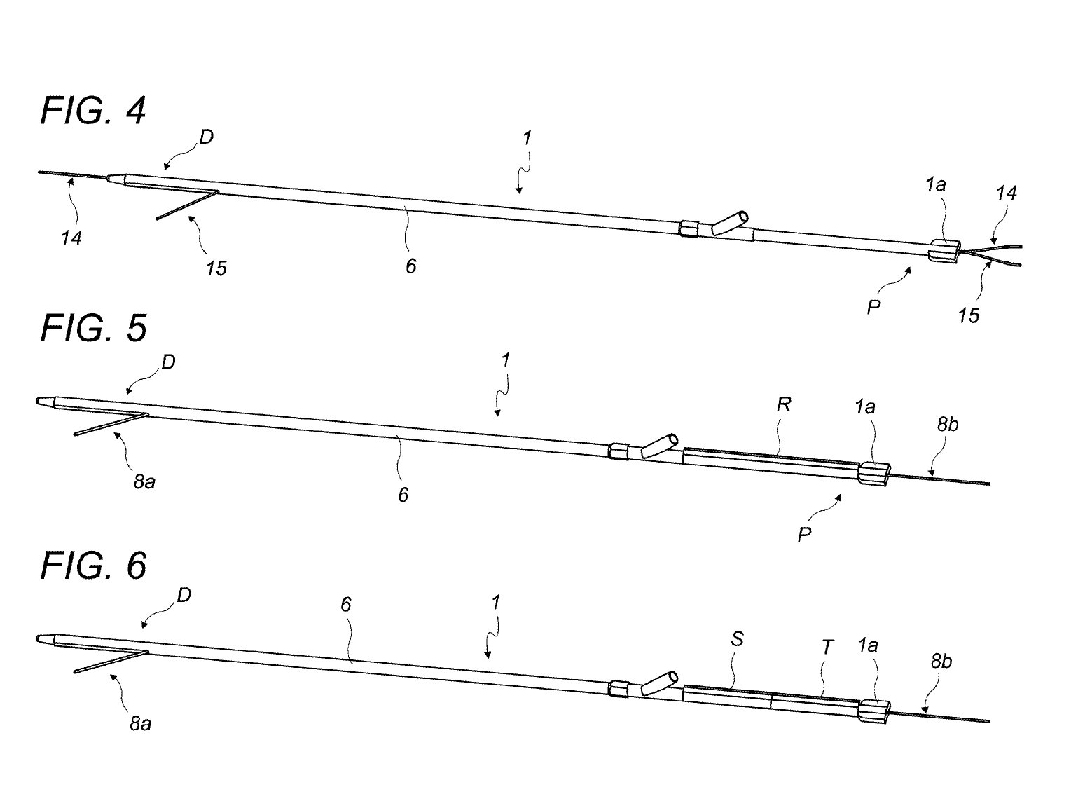

Also in reference to FIGS. 4 and 13, it appears that the assembly also comprises a main guide 14 pre-implantable in the main conduit C1 of the patient and intended to engage in the axial hole 5 for guiding purposes of the main driver 1.

The assembly also includes a secondary guide 15 pre-implantable in the main conduit C1 and in a secondary conduit C2 while passing through the bifurcation formed by said conduits C1, C2, to engage in the complementary sheath 8 for positioning purposes of the main driver 1. Furthermore, said secondary guide 15 makes it possible to guide the secondary driver 2 for positioning of the secondary implant 13. Such guides 14, 15 are known as such and will therefore not be described further.

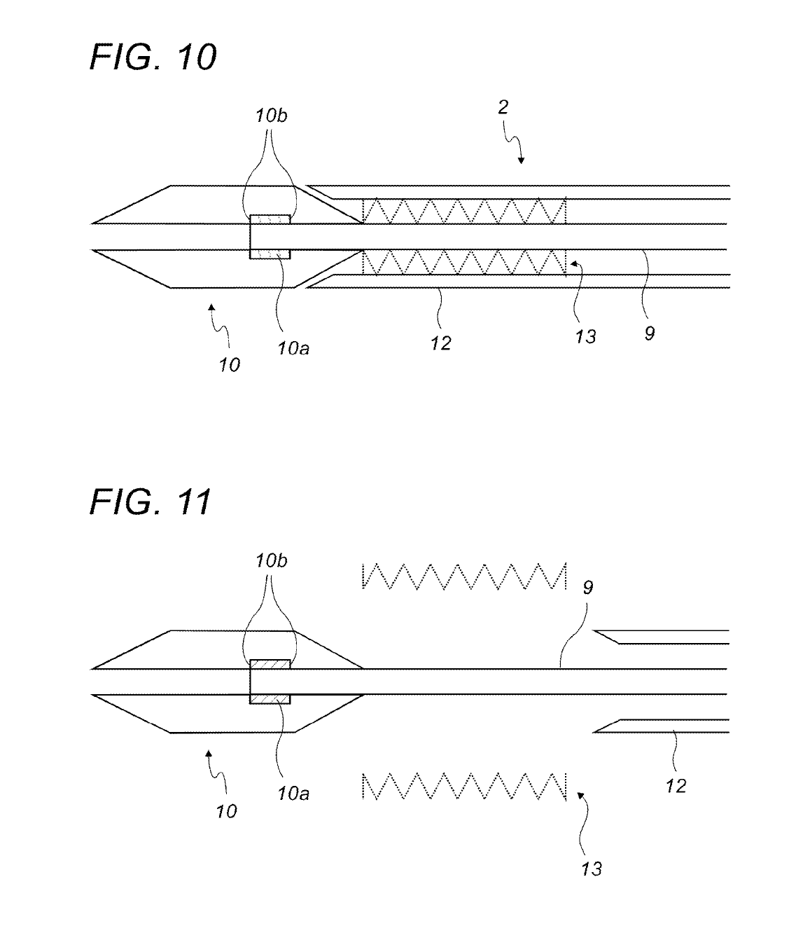

FIG. 10 is an enlarged schematic illustration of a detail of the secondary driver 2 of FIGS. 7, 8 and 9, in which the secondary implant 13, which may also be called branch implant, is disposed before insertion, non-deployed between the external 12 and internal 9 secondary sheaths.

FIG. 11 illustrates the detail of FIG. 10 with the secondary implant 13, for example self-expandable, released and deployed in situ in the secondary conduit C2, after the sliding of the external sheath 12 toward the fixed point 1b of the secondary driver 2.

According to one advantageous embodiment of the assembly, the secondary atraumatic tip 10 of the secondary driver 2 is a dual-cone tip. Such a configuration substantially reduces the risks of catching of the implants during the handling of the secondary driver 2, and in particular during the removal thereof from the secondary conduit C2.

The secondary atraumatic tip 10 is maintained on the secondary internal sheath 9 using a ring 10a secured to said secondary internal sheath 9. The ring 10a is for example fastened on the secondary internal sheath 9 by gluing, heat shrinking or any other known means. The secondary atraumatic tip 10 also has an internal housing delimiting shoulders 10b between which the ring 10a is housed, preventing any sliding on the secondary internal sheath 9 and as a result preventing a separation.

According to one example embodiment, illustrated for example in FIG. 5, the main external sheath 6 is mounted sliding on the internal sheath 3 and blocked in sliding by the atraumatic tip 4 on the one hand and by a removable stop R on the other hand.

According to another example embodiment, illustrated in FIG. 6, the main external sheath 6 is mounted sliding on the internal sheath 3 and blocked in sliding by the atraumatic tip 4 on the one hand and by two successive removable stops S, T, on the other hand.

The stops R or S and T are blocked in sliding between the proximal end of the main external sheath 6 and the fixed point 1a.

According to one example embodiment, the secondary external sheath 12 is mounted sliding on the secondary internal sheath 9 and blocked in sliding by the secondary atraumatic tip 10 on the one hand and by three successive and removable stops A, B, C on the other hand.

The stops A, B, C are blocked in sliding between the proximal end of the secondary external sheath 12 and the fixed point 1b.

The successive stops A, B and C provide, by their separation from the secondary driver 2, a gradual release of the secondary implant 13 in the secondary conduit C2 and as a result its connection on the main implant 7 housed in the main conduit C1.

According to another example embodiment, not shown in the figures, the secondary external sheath 12 is mounted sliding on the secondary internal sheath 9 and blocked in sliding by the secondary atraumatic tip 10 on the one hand and by two successive A, B and removable A, B stops on the other hand.

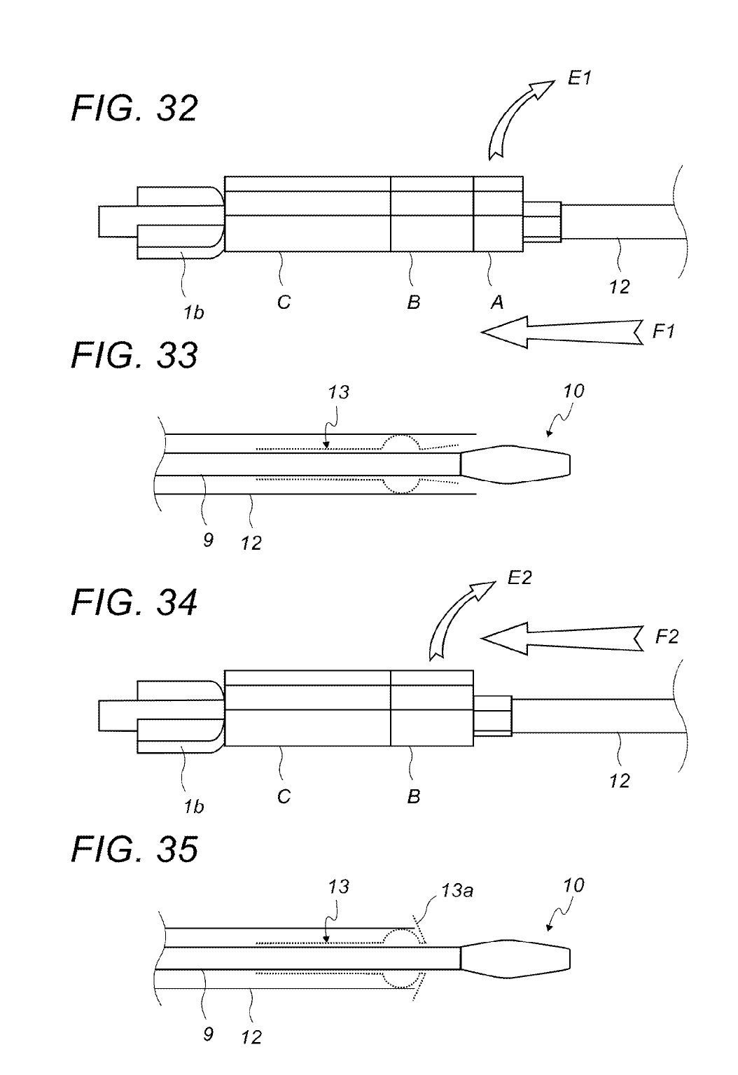

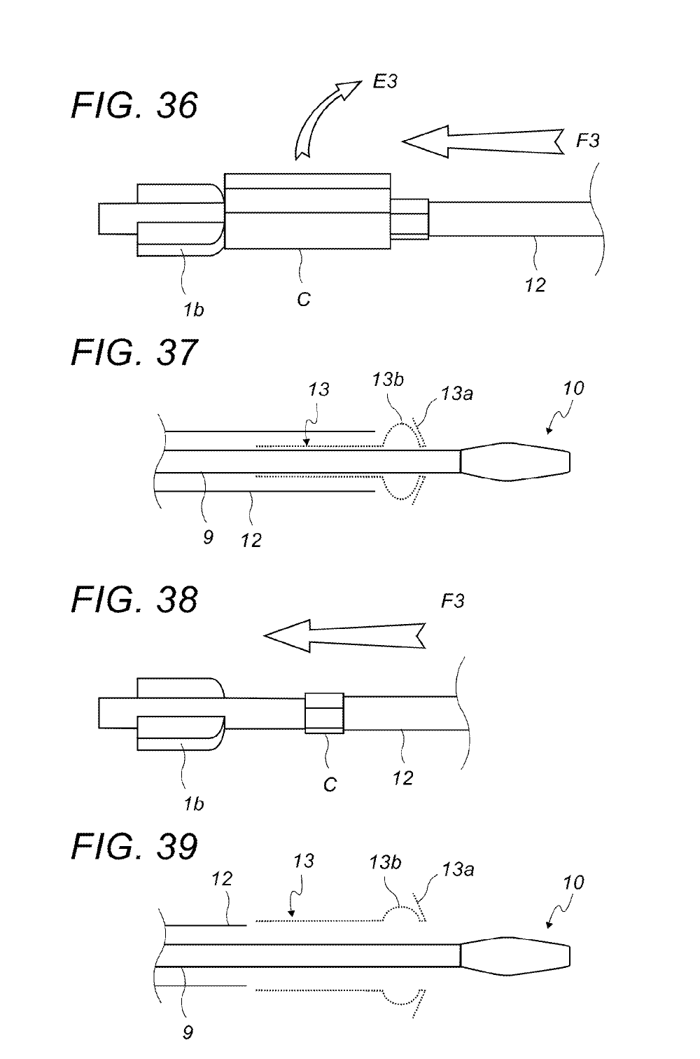

According to one example embodiment, the secondary implant 13 is configured to have connecting members 13a, 13b cooperating with the main implant 7. The connecting members 13a and 13b automatically perform the connection with the main implant 7 during the release and deployment of said secondary implant 13. Reference may also be made to FIGS. 32 to 39.

According to one example embodiment, the complementary sheath 8 is advantageously extractable from the main driver 1. The complementary sheath 8 is thus removed from the main driver 1 when the secondary guide 15 is placed in the conduits C1, C2 and in the main driver 1 by passing through said complementary sheath 8. The latter allows the secondary guide 15, also called lateral guide, to be introduced into the main driver 1 and in particular to ensure the easy passage in the window 7a of the non-deployed main implant 7 and the easy passage of the lateral hole 3a of the internal sheath 3. This operation can thus be done quickly with great reliability.

According to one example embodiment, not shown in the figures, the secondary driver 2 has a curved secondary atraumatic tip 10. Such a shape makes it possible to better orient the direction of approach of the secondary implant 13 with respect to the window 7a arranged in the main implant 7. This may prove useful for certain shapes of bifurcations and for certain curve radii encountered.

According to one exemplary embodiment, the secondary driver 2 advantageously has an end portion made with a flexible material. Advantageously, this end portion may have a curved shape.

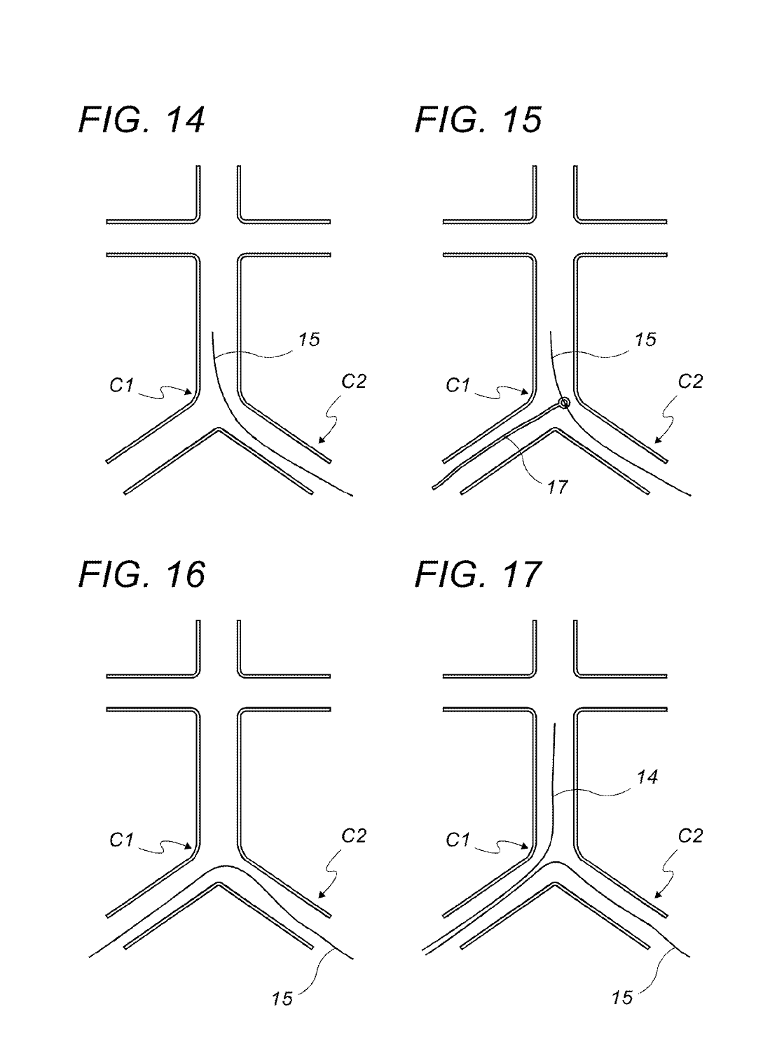

The assembly makes it possible to carry out a new method for inserting implants 7, 13 that is particularly simple, precise and reliable. Indeed, the insertion method makes it possible to ensure that the implants 7 and 13 are positioned and oriented correctly inside the conduits C1 and C2. This encourages a quick and reliable connection of said implants 7 and 13 to one another. Schematic FIGS. 14 to 25, FIGS. 26 to 31 and FIGS. 32 to 39 illustrate an example embodiment of one such new method.

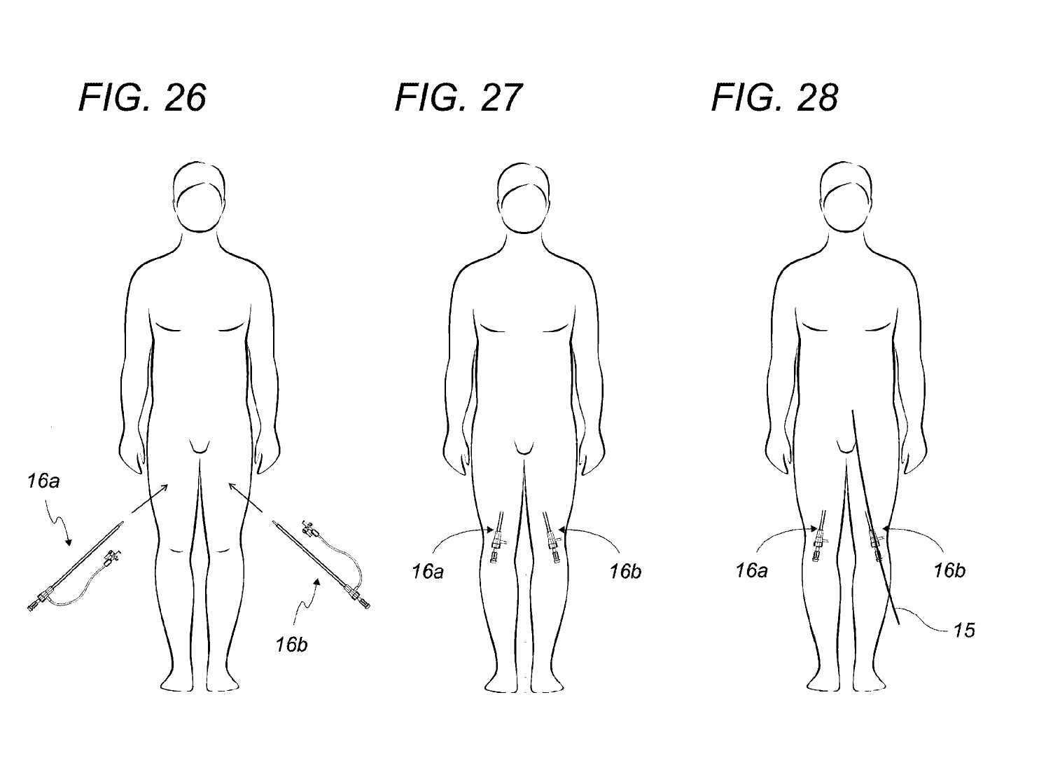

The steps relating to the preparation of the patient are not described in detail in this document. These preparatory steps are in fact widely known. They in particular involve inserting introducers 16a and 16b into the patient. Reference can be made to FIGS. 26 and 27.

Thus, the method carried out using the assembly consists of engaging the secondary guide 15, which may also be called lateral guide, in the secondary conduit C2 using an introducer 16b. Reference can be made to FIGS. 14 and 28.

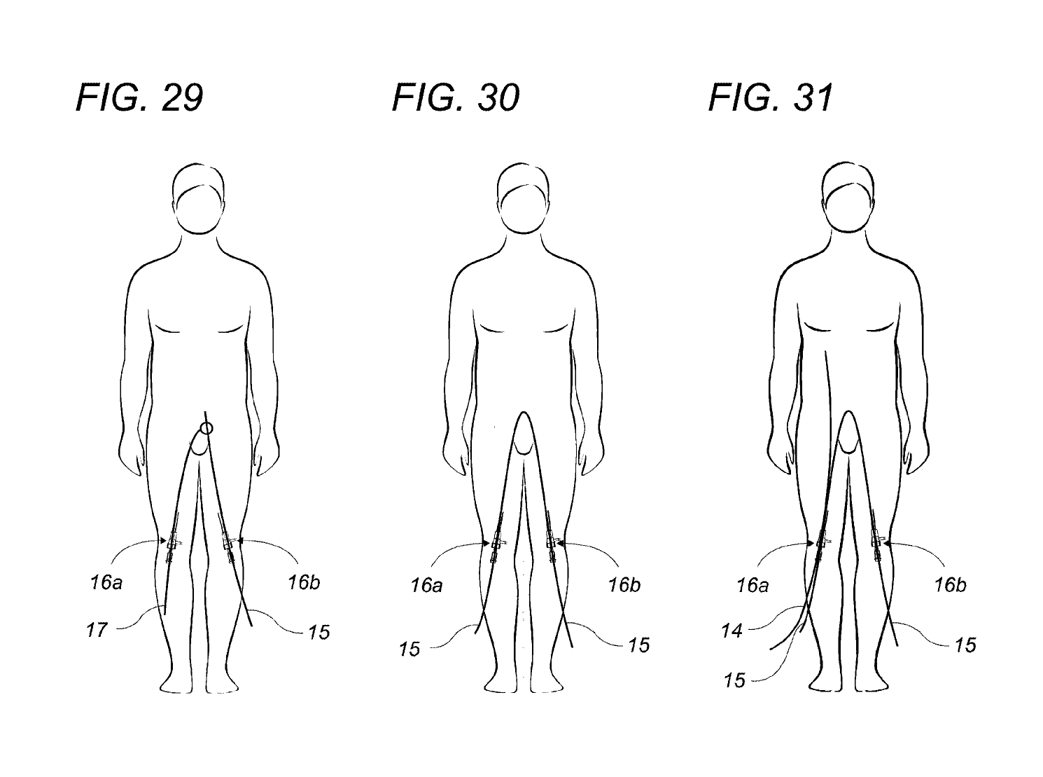

Next, the method consists of introducing, into the main conduit C1, via another introducer 16a, a gripping member 17, of the lasso type, to recover the secondary guide 15 at the bifurcation between the conduits C1 and C2. Reference can be made to FIGS. 15 and 29.

The gripping member 17 thus makes it possible to hook the secondary guide 15 and cause it to pass into the main conduit C1 and extract it therefrom by one of its ends at the other introducer 16a. Reference can be made to FIGS. 16 and 30.

FIGS. 17 and 31 illustrate the positioning of the secondary guide 15, which has a first portion extending in the main conduit C1 and leaving the latter with one of said ends via the other introducer 16a. The secondary guide 15 also extends in the secondary conduit C2 and leaves the latter with the other of said ends via the introducer 16b.

FIGS. 17 and 31 also illustrate a complementary step consisting of engaging the main guide 14, which may also be called axial guide, in the main conduit C1 using the other introducer 16a.

The end of the secondary guide 15 leaving through an incision on the main conduit C1 side and through the other introducer 16a is next engaged in the first end 8a of the complementary sheath 8 of the main driver 1.

The main guide 14, and more specifically its end leaving the other introducer 16a, is next engaged in the main driver 1 through the axial hole 5.

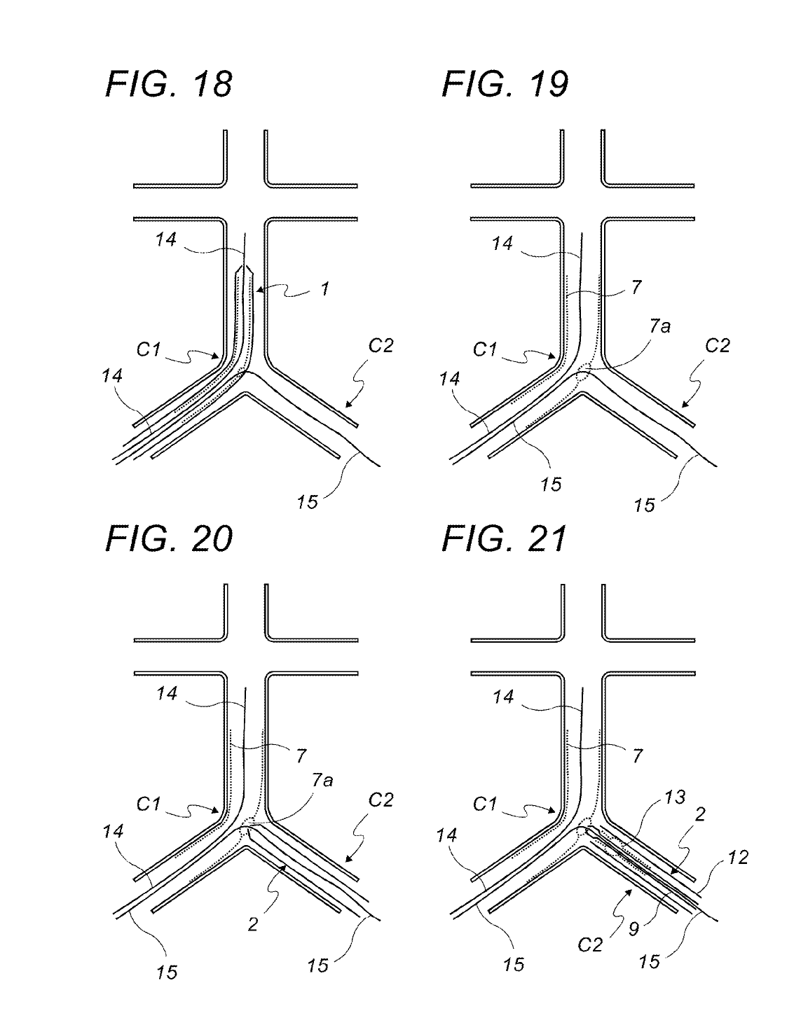

The complementary sheath 8 is next removed from the main driver 1. The latter is next introduced into the main conduit C1, via the other introducer 16a, to be positioned at the bifurcation such that the lateral hole 3a and the window 7a of the main implant 7 are found substantially in the extension of the secondary conduit C2. This position is easily identifiable during the guiding of the main driver 1 by the main guide 14, owing to the radiopaque localization of the secondary guide 15. The main guide 14 primarily makes it possible to guide the advance of the main driver 1 in the main conduit C1. Reference may in particular be made to FIG. 18.

FIG. 19 illustrates the step during which the external sheath 6 is slid toward a fixed point 1a of the main driver 1 so as to release the main implant 7. The latter thus deploys at the bifurcation. The release of the main implant 7 is obtained for example by the removal of a stop R of the main driver 1 of FIG. 5. According to another example embodiment, the release of the main implant 7 is obtained by the successive removal of the two adjacent stops S, T of the main driver 1 illustrated in FIG. 6.

Once the main implant 7 is fully deployed, as illustrated in FIG. 19, the main driver 1 is removed from the main conduit C1.

Next, during an additional step, the method consists of introducing, via the introducer 16b, the secondary driver 2, guided by the secondary guide 15, into the secondary conduit C2 up to the level of the window 7a arranged in the main implant 7. This window 7a is of course located at the bifurcation and is passed through by the secondary guide 15. Reference may be made to FIG. 20.

FIGS. 21 to 25 and 32 to 39 then illustrate the release of the secondary implant 13 as well as its connection on the main implant 7 during successive operations.

Thus, FIGS. 21 and 33 illustrate the positioning of the secondary driver 2 with the secondary external sheath 12, the secondary internal sheath 9, the secondary implant 13 and the secondary guide 15 before deployment and release of said secondary implant 13.

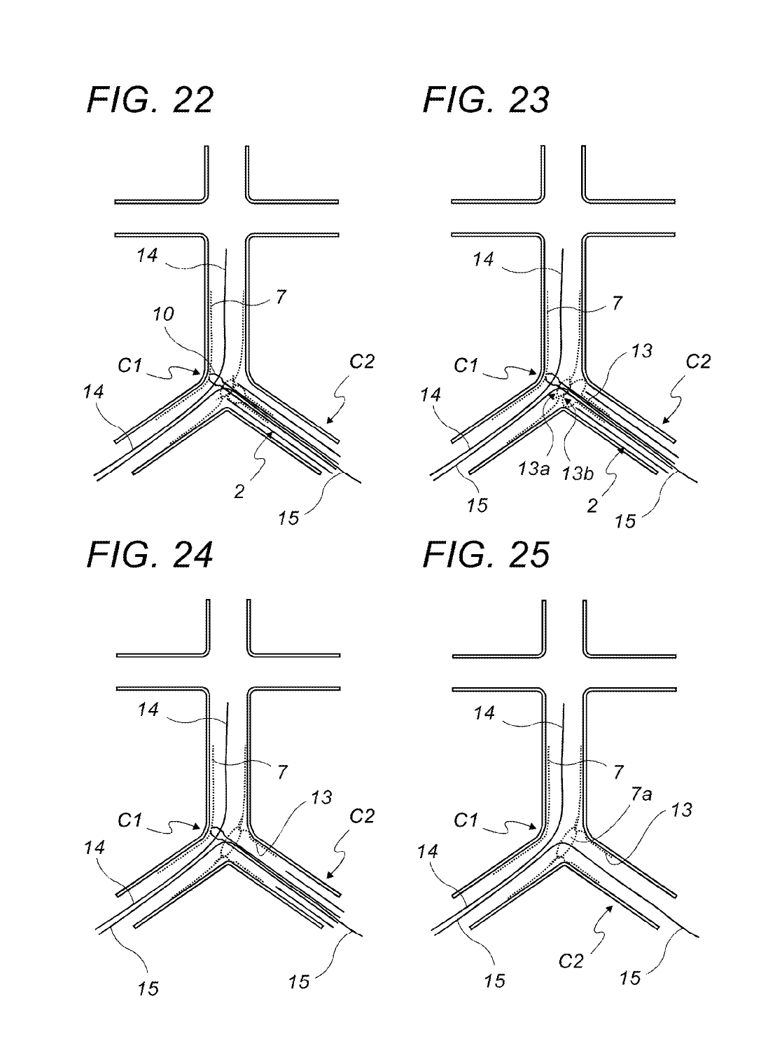

The method next consists of removing the first stop A from the secondary driver 2 (arrow E1) and sliding (arrow F1) the secondary external sheath 12 against the second stop B of said secondary lateral driver 2. This makes it possible to release a first end of the secondary implant 13 and in particular the connection members 13a beyond the window 7a of the main implant 7. This step is also illustrated with FIGS. 22 and 35. The connection members 13a are for example made up of a foldable end portion 13a of the secondary implant 13.

Next, according to an additional step, the method consists of removing the second stop B from the secondary driver 2 (arrow E2) and sliding (arrow F2) the secondary external sheath 12 up to the third stop C so as to release also a positioning bead 13b of the secondary implant 13.

This bead 13b is, however, located before the window 7a of the main implant 7. The bead 13b and the foldable end portion 13a thus make it possible to block the secondary implant 13 on the main implant 7 at the window 7a. A partial obstruction of the main conduit C1 by an end of said secondary implant 13 is thus avoided. The latter indeed cannot protrude into the main implant 7. This step is also illustrated for example with FIGS. 23 and 37.

The method next consists of removing the third stop C (arrow E3) and sliding (arrow F3) the secondary external sheath 12 toward the fixed point 1b of the secondary driver 2. This corresponds to a release and a complete deployment of the secondary implant 13 as well as a connection of the latter on the main implant 7 at the window 7a. Reference can for example be made to FIGS. 24, 36, 38 and 39.

A complementary step consists of removing the main guide 14 and the secondary guide 15 from the respective conduits C1 and C2.

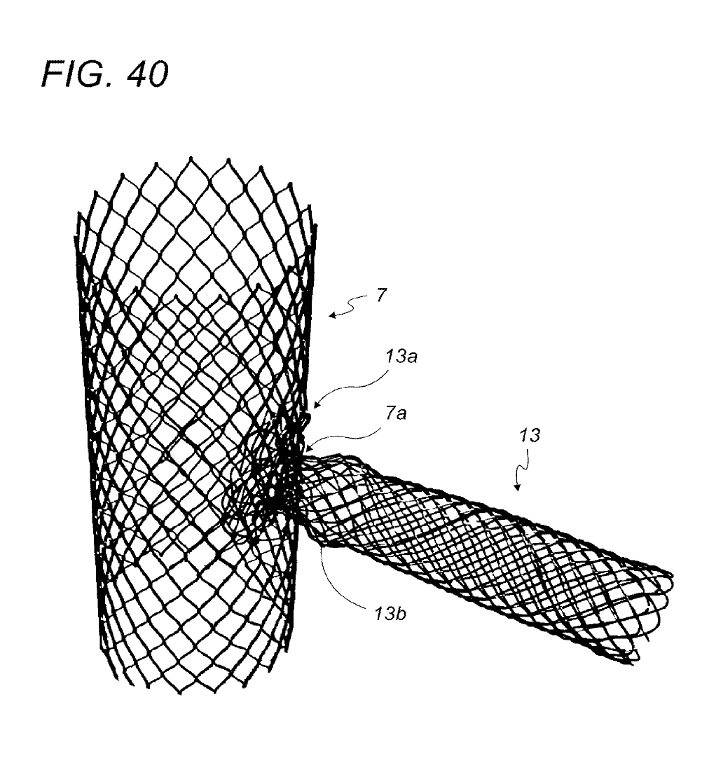

One thus obtains a secondary implant 13 and a main implant 7 that are connected at a bifurcation between a main conduit C1 and a secondary conduit C2, as for example shown in FIGS. 25 and 40.

Obviously, the following claims are not limited to the preferred embodiment or to the implementation described above and shown in the various figures, a person skilled in the art being able to make numerous modifications and imagine other example embodiments and/or implementations. Thus, a described technical characteristic or a described implementation step can be replaced by an equivalent technical characteristic, respectively an equivalent step, without leaving the framework or scope of the contemplated embodiments.

1. A main driver having at least one main implant for the insertion of said at least one main implant connectable to another implant, at a bifurcation between a main conduit and a secondary conduit of circulation for a bodily fluid, said main driver having a distal end D and a proximal handling end P having a fixed point,
wherein:
said driver has a main internal sheath provided at a distal end D thereof with an atraumatic tip with an axial hole communicating with a longitudinal tube formed by said internal sheath,
said driver having a main external sheath,
said at least one main implant being disposed between the main external and internal sheaths,
the main external sheath being provided with a longitudinal groove or slit extending along a part of a length of the main external sheath,
the main internal sheath having a lateral hole emerging facing said longitudinal groove or slit,
the main implant having at least one window aligned with the lateral hole,
and said driver having at least one complementary sheath, a first end of which passes through the lateral hole, the window of the main implant and the longitudinal groove or slit, and a second end of which comes out near the proximal end P, said complementary sheath being intended to be passed through by a guide.

2. The driver according to claim 1, wherein the main external sheath is mounted sliding on the main internal sheath and blocked in sliding by the atraumatic tip on the one hand and by at least one removable stop on the other hand, said longitudinal groove or slit being open at a distal end D thereof near the atraumatic tip.

3. The driver according to claim 1, wherein the main external sheath is made of a peelable material.

4. The driver according to claim 1, wherein the complementary sheath is extractable from the driver and allows the insertion of a secondary guide into said driver and makes it possible to ensure the passage in the window of the main implant and the passage of the lateral hole of the main internal sheath.

5. An assembly for the insertion and the connection of at least two implants in a main conduit and secondary conduit of circulation for a bodily fluid, the main conduit having a bifurcation with the secondary conduit, further comprising:
a main driver according to claim 1,
a secondary driver, independent of the main driver, having a distal end D and a proximal handling end P having a fixed point,
said secondary driver having an internal secondary sheath provided at a distal end D thereof with a secondary atraumatic tip with a secondary axial hole communicating with a longitudinal tube formed by said internal secondary sheath, an external secondary sheath, at least one secondary implant disposed between the external and internal secondary sheaths.

6. The assembly according to claim 5, further comprising a main guide pre-implantable in the main conduit and intended to engage in the axial hole for guiding purposes of the main driver.

7. The assembly according to claim 5, further comprising a secondary guide preimplantable in the main conduit and in the secondary conduit while passing through the bifurcation, in order to engage in the complementary sheath for positioning purposes of the main driver and/or for guiding purposes of the secondary driver.

8. The assembly according to claim 5, wherein the secondary atraumatic tip of the secondary driver is a double-cone tip.

9. The assembly according to claim 5, wherein the secondary external sheath is mounted sliding on the secondary internal sheath and blocked in sliding by the secondary atraumatic tip on the one hand and by at least two successive and removable stops on the other hand, said stops ensuring the gradual release of the secondary implant as well as connection of the secondary implant on the main implant.

10. The assembly according to claim 9, wherein the secondary external sheath is mounted sliding on the secondary internal sheath and blocked in sliding by the secondary atraumatic tip on the one hand and by three successive and removable stops on the other hand.

11. The assembly according to claim 5, wherein the secondary implant is configured to have connection members to the main implant, which automatically form the connection with the main implant during the release and deployment of said secondary implant.

12. The assembly according to claim 5, wherein the secondary atraumatic tip has a curved distal end D.

13. The assembly according to claim 5, wherein the secondary atraumatic tip is made from a flexible material.

14. The assembly according to claim 5, wherein the implants are connected to the corresponding internal sheath by means of a retaining member making it possible to remove and/or reposition said implants in the driver if needed during the insertion operation.

15. A method for the insertion and in situ connection of at least two implants in a main conduit and a secondary conduit of circulation for a bodily fluid, the main conduit of circulation having, with the secondary conduit of circulation, a bifurcation, said method being carried out with an assembly according to claim 5, further comprising the following steps:
engaging a secondary guide in the secondary conduit,
introducing a gripping member into the main conduit and attaching the secondary guide at the level of the bifurcation,
passing the end of the secondary guide in the main conduit and extracting one end thereof from the main conduit,
engaging a main guide in the main conduit,
engaging the end of the secondary guide, exiting from the main conduit, into the first end of the complementary sheath of the main driver,
engaging the end of the main guide, exiting from the main conduit, into the axial hole of main driver,
removing the complementary sheath from the main driver,
introducing the main driver and positioning the main driver at the bifurcation using the main guide,
sliding the external sheath over the internal sheath to release the main implant and deploy the main implant,
introducing the secondary driver into the secondary conduit up to the level of the main implant using the secondary guide,
sliding the external secondary sheath over the internal secondary sheath to gradually release the secondary implant, deploy the secondary implant, and connect the secondary implant to the main implant,
removing the main and secondary drivers as well as the main and secondary guides from the conduits.

16. The method according to claim 15, further consisting of of sliding the external secondary sheath and using a system of removable stops to block the sliding of the external secondary sheath in successive positions corresponding to the gradual release of the secondary implant as well as connection of the secondary implant to the main implant.

17. The method according to claim 16, further consisting of repositioning the secondary implant in the secondary driver for a release and placement in another more suitable location in the secondary conduit.
